(19) United States
(12) Patent Application Publication
(10) Pub. No.: US 2005/0196348 A1
(43) Pub. Date: Sep. 8, 2005

(54) Stabilized peroxide compositions

(76) Inventors: Constantine Georgiades, East Brunswick, NJ (US); Seema Mody, Montville, NJ (US); Andre Soshinsky, Randolph, NJ (US); Zhen Zhang, Basking Ridge, NJ (US)

Correspondence Address: PFIZER, INC., 201 TABOR ROAD, MORRIS PLAINS, NJ, 07950, US

(21) Appl. No.: 10/792,370

(22) Filed: Mar. 3, 2004

(51) Int. Cl. A61K 9/68; A61K 7/20

(52) U.S. Cl. 424/48; 424/53

(57) ABSTRACT

This invention relates to a stabilized, oral composition, and more particularly, to such composition comprising peroxide compounds.

### FIELD OF THE INVENTION

This invention relates to a stabilized, oral composition, and more particularly, to such composition comprising peroxide compounds.

### BACKGROUND OF THE INVENTION

Hydrogen peroxide is a well known antiseptic which has been extensively employed in aqueous solution for the treatment of infectious processes in both human and veterinary topical therapy. The agent can be used in its original form after suitable dilution, or it can be derived from those solid compounds which form salts or additive compounds with hydrogen peroxide. Included among these are sodium perborate, sodium carbonate peroxide, sodium peroxyphosphate, urea peroxide, potassium persulfate, and others. When added to water, these compounds hydrolyze into hydrogen peroxide and the corresponding carrying salt.

Although extensively employed for treating all parts of the body, hydrogen peroxide has proved especially valuable for treating the mucous membranes of the oral cavity. Partly as a consequence of oxygen tissue metabolic and reparative requirements (by a mechanism which is not clearly understood), partly as a consequence of its broad antibacterial effects against gram positive and gram negative cocci, bacillus and spirochetal forms as well as many varieties of yeasts and fungi, and partly because of its cleaning and hemostatic effects, hydrogen peroxide is extensively recommended and used for bacterial and viral infections and for tissue inflammations of non-microorganic origin.

The principal limitations of commonly used peroxide aqueous solutions, however, are their poor shelf stability caused by the decomposition of hydrogen peroxide into gaseous oxygen and water at room temperature, and the transitory contact of the active oxygenating agent with the affected tissue. In addition, when such compositions are formed of additive compounds with hydrogen peroxide, it is common to prepare the adduct composition before incorporating it into the desired composition.

Attempting to address this limitation, the inventors of the present invention have discovered that oral compositions incorporating at least two water soluble gums in addition to the peroxide component have improved peroxide stability.

Accordingly an aspect of the present invention is to provide improved oral compositions.

Another aspect of the present invention is to provide oral compositions providing improved peroxide stability.

Still one other aspect of the present invention is to provide oral compositions comprising at least two water-soluble gums and a peroxide compound having improved peroxide stability.

Still yet one other aspect of the present invention is to provide improved oral teeth whitening compositions, especially films.

These and other objects and features of the invention will be made apparent from the following description thereof.

### SUMMARY OF THE INVENTION

The present invention relates to oral compositions or solid oral compositions comprising at least two water soluble gums and a peroxide compound wherein the composition, when dried, contains less than about 10% of an hydroalcoholic component. The active, oxygen-generating peroxide component in the composition is stabilized in the oral composition.

### DETAILED DESCRIPTION OF THE PRESENT INVENTION

The oral compositions of the present invention can comprise, consist of, or consist essentially of the essential elements and limitations of the invention described herein, as well any of the additional or optional ingredients, components, or limitations described herein.

All percentages, parts and ratios are based upon the total weight of the oral composition of the present invention prior to drying, unless otherwise specified. All such weights as they pertain to listed ingredients are based on the active level and, therefore, do not include carriers or by-products that may be included in commercially available materials, unless otherwise specified.

The term “safe and effective amount” as used herein means an amount of a compound or composition such as a topical or system active sufficient to significantly induce a positive benefit, for example, a teeth whitening, antimicrobial and/or analgesic benefit, including independently the benefits disclosed herein, but low enough to avoid serious side effects, i.e., to provide a reasonable benefit to risk ratio, within the scope of sound judgment of the skilled artisan.

The term “adhesive” as used herein, means any material or composition that is capable of sticking to the site of topical application or administration and includes, but is no limited to, mucoadhesives, pressure-sensitive adhesive (adheres upon application of pressure), moistenable adhesives (adheres in the presence of water) and tachy or sticky type adhesives (adheres upon immediate contact with a surface).

The term “foreign substances” as used herein means dirt, infectious microorganisms and the like.

Optionally, the film compositions of the present invention are clear. The term “clear” as defined herein ranges from transparent to translucent as observed with the naked eye.

The film compositions of the present invention, including the essential and optional components thereof, are described in detail hereinafter.

Essential Ingredients

Water soluble Gums

The compositions of the present invention preferably contain a water-soluble, synthetic or natural gum. Gums suitable for use herein include, but are not limited to algin, alginic acid, alginate salts, camitine, carrageenan, karaya gum, dextrin (starch gum), guar gum, gellan gum, irish moss, veegum (regular), tara gum, okra gum, gum arabic, acacia gum, amylopectin, pectina or pecitin, ghatti gum, natto gum, tragacanth gum, xanthan gum, sclerotium gum, kelp, locust bean gum, psyllium seed, tamarind gum, destria gum, chitosan, esters thereof (such as hydroxypropyl chitosan and hydroxypropyl guar), salts thereof (such as ammonium alginate, amylopectin, calcium alginate, calcium carrageenan, guar hydroxypropyltrimonium), and mixtures thereof. Additional gums or salts or derivatives thereof can be found in U.S. Pat. No. 6,551,604 to Beck et al. which is herein incorporated by reference in its entirety.

In certain embodiments, the water soluble gum can includes xanthan gum (supplied by CP Kelco, Chicago, Ill.), locust bean gum (supplied by Degussa Texturant System, Atlanta, Ga.), carrageenan (supplied by FMC Biopolymer, Philadelphia, Pa.) or mixtures thereof.

When incorporated into the oral compositions of the present invention, the water soluble gum is present at a concentration of from about 0.01% to about 10% optionally, from about 0.1% to about 5% optionally, from about 0.1% to about 1%, and, optionally, from about 0.1% to about 2%, by weight, of the wet film composition.

Peroxide Compounds

Also incorporated into the compositions of the present invention is a peroxide compound. Suitable peroxide compounds include, but is not limited to, hydrogen peroxide, calcium peroxide, urea peroxide, carbamide peroxide, and mixtures thereof. In certain embodiments, the peroxide is hydrogen peroxide.

The effectiveness of peroxide materials can, optionally, be enhanced by means of a catalyst, i.e. a two-component peroxide-catalyst system. Useful peroxide catalysts or catalytic agents can be found in U.S. Pat. No. 6,440,396 to McLaughlin, Gerald, herein incorporated by reference in its entirety.

When incorporated into the oral compositions of the present invention, the peroxide is present at a concentration of from about 0.1% to about 20%, optionally from about 0.5% to about 15% optionally, from about 1% to about 12%, and, optionally, from about 5% to about 10%, by weight, of the wet film composition.

In certain embodiments, the ratio of the water-soluble gum to the peroxide component is at least about 1:25, optionally, from about 1:25 to about 1:5, optionally, from about 1:20 to about 1:7 and, optionally, from about 1:16 to about 1:10.

Hydroalcoholic component.

The solid oral compositions of the present invention, when dried, are substanitially free of hydroalcoholic components. As used herein the term “hydroalcoholic” means water or alcohol or mixtures thereof. In certain embodiments of the present invention, the compositions of the present invention contain less than 10% (or about 10%), optionally, less than 6% (or about 6%), and, optionally, less than 3% (or about 3%) of an hydroalcoholic component.

Optional Ingredients

Various other actives, especially oral care actives, can also be incorporated into the oral compositions of the present invention. Examples of the conditions these actives may address include, but are not limited to one or more of, appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores, tooth abscesses, tooth and/or gum pain, tooth sensitivity (e.g. to temperature changes), teeth strengthening and the elimination of mouth malodour resulting from the conditions above and other causes such as microbial proliferation. Additionally, the films of the present invention are useful for treating and/or preventing wounds, lesions, ulcers, cold sores and the like of the lips and skin generally.

Suitable topical actives for use in and around the oral cavity include any substance that is generally considered as safe for use in the oral cavity and that provides a change to the overall health of the oral cavity. The level of topical oral care active in the present invention may generally be from about 0.01% to about 40% or, optionally, from about 0.1% to 20% by weight of the wet film.

The topical oral care actives of the present invention may include many of the actives previously disclosed in the art. The following is a non all-inclusive list of oral care actives that may be used in the present invention.

Essential oils may be included in or associated with the films the present invention. Essential oils suitable for use herein are described in detail in U.S. Pat. No. 6,596,298 to Leung et al., previously incorporated by reference in its entirety.

Additional teeth whitening actives may be included in the oral compositions of the present invention. The additional actives suitable for whitening are selected from the group consisting of metal chlorites, perforates, percarbonates, peroxyacids, and mixtures thereof. Suitable metal chlorites include calcium chlorite, barium chlorite, magnesium chlorite, lithium chlorite, sodium chlorite and potassium chlorite. Hypochlorite and chlorine dioxide may also be incorporated into the compositions of the present invention. A preferred chlorite is sodium chlorite.

Anti-tartar agents useful herein include: phosphates. Phosphates include pyrophosphates, polyphosphates, polyphosphonates and mixtures thereof. Pyrophosphates are among the best known for use in dental care products. Pyrophosphate ions delivered to the teeth derive from pyrophosphate salts. The pyrophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are preferred. Anticalculus phosphates include potassium and sodium pyrophosphates; sodium tripolyphosphate; diphosphonates, such as ethane-1-hydroxy-1,1-diphosphonate; 1-azacycloheptane-1,1-diphosphonate; and linear alkyl diphosphonates; linear carboxylic acids and sodium and zinc citrate.

Agents that may be used in place of or in combination with the pyrophosphate salt include materials such as synthetic anionic polymers including polyacrylates and copolymers of maleic anhydride or acid and methyl vinyl ether (e.g. Gantrez, as described, for example, in U.S. Pat. No. 4,627,977, to Gaffar et al. herein incorporated by reference in its entirety, as well as e.g. polyamino propane sulfonic acid (AMPS), zinc citrate trihydrate, polyphosphates (e.g. tripolyphosphate; hexametaphosphate), diphosphonates (e.g. EHDP, AMP), polypeptides (such as polyaspartic and polyglutamic acids), and mixtures thereof.

One of more fluoride ion sources incorporated into the film compositions as anticaries agents. Fluoride ions are included in many oral care compositions for this purpose, and similarly may be incorporated in the invention in the same way. Detailed examples of such fluoride ion sources can be found in U.S. Pat. No. 6,121,315 to Nair et al., herein incorporated by reference in its entirety.

Antimicrobial agents can also be present in the film compositions of the present invention as oral agents or topical skin and/or systemic actives. Such agents may include, but are not limited to, 5-chloro-2-(2,4-dichlorophenoxy)-phenol, commonly referred to as triclosan, chlorhexidine, alexidine, hexetidine, sanguinarine, benzalkonium chloride, salicylamide, domiphen bromide, cetylpyridium chloride (CPC), tetradecyl pyridinium chloride (TPC); N-tetradecyl-4-ethyl pyridinium chloride (TDEPC); octenidine; delmopinol, octapinol, and other piperidino derivatives, niacin preparations; zinc/stannous ion agents; and analogs, derivatives and salts of the above antimicrobial agents and mixtures thereof.

Anesthetic agent may also be incorporated herein. Examples of suitable anesthetic agents include, but are not limited to, benzocaine, betoxycaine, biphenamine, bupivacaine, butacaine, dibucaine hydrochloride, dyclonine, lidocaine, mepivacaine, procaine, propanidid, propanocaine, proparacaine, propipocaine, propofol, propoxycaine hydrochloride, pseudococaine, tetracaine hydrochloride and mixtures thereof.

Additional useful actives can be found in U.S. Pat. No. 6,638,528 herein incorporated by reference in its entirety.

An additional carrier material may also be added to the oral care film composition. These materials are generally humectants and include glycerin, sorbitol, polyethylene glycol and the like. The oral healthcare film may comprise the active substance itself, together with one or more active substance enhancers, for example catalysts and/or potentiators to modify the release and/or activity of the active substance.

The film compositions of the invention may, optionally, comprise additional substances such as flavors, colors etc. which may for example be deposited onto the surface of the film or impregnated into the bulk of the film.

For example a gel containing additional substances or actives may be deposited directly as a layer on a surface of a film layer. Alternatively additional substances or actives may be absorbed into the above-described film layer, or impregnated into the bulk of the film material, or deposited between layers of a multiple layered film.

A pH adjusting agent may also be added to optimise the storage stability of the gel and to make the substance safe for the oral tissues. These pH adjusting agents, or buffers, can be any material which is suitable to adjust the pH of the oral care substance. Suitable materials include sodium bicarbonate, sodium phosphate, sodium hydroxide, ammonium hydroxide, sodium stannate, triethanolamine, citric acid, hydrochloric acid, sodium citrate, and combinations thereof. The pH adjusting agents are added in sufficient amounts so as to adjust the pH of the substance or composition to a suitable value, e.g. about 4.5 to about 11, preferably from about 5.5 to about 8.5, and more preferably from about 6 to about 7. The pH adjusting agents are generally present in an amount of from about 0.01% to about 15% and preferably from about I 0.05% to about 5%, by weight of the oral care substance.

Methods of depositing active substances mentioned above or additional actives upon the surfaces of film materials are known, for example printing, e.g. silo screen printing, passing between impregnated rollers, dosing, a pump and nozzle, spraying, dipping etc. Methods of impregnating substances into the bulk of film materials are also known, for example admixing the substance into the strip material and then forming the strip, or exposure of the strip to the substance under conditions which cause the substance to be impregnated into the strip. Alternatively, one example of the film material may be a foam material, particularly an open-cell foam material, and the substance may be impregnated into the strip material by introducing the substance into the cells of the foam.

The device of the invention may be marked with one or more visible symbol, e.g. text matter, a trade mark, a company logo, an area of color, or an alignment feature such as a visible line or notch etc. to assist the user in applying the device to the teeth in a proper alignment. Such an alignment feature may for example comprise a symbol to show the user which way up the device should be whilst applying the device to the teeth, or which of a pair of the devices is intended for the upper teeth and which for the lower teeth. This way the device may be made more visually attractive and/or easier to use. Such symbol(s) may be applied by conventional printing or embossing processes, e.g. silk screen printing, inkjet printing etc. to the surface of the plastically deformable material opposite to the surface on which is attached the layer of an absorbent material.

If such a visible symbol is applied to this surface, a cover layer can, optionally, be applied over the symbol, for example to protect it. This cover layer may be transparent or translucent to allow visible symbols to be seen through this layer. Such a cover layer can, optionally, be applied to the film by pressing, e.g. rolling, the material of the cover layer in contact with the film.

Methods for Delivering Topical and Systemic Actives

The present invention can be used where retention of peroxides and additional topical or systemic actives is required for topical activity or adequate systemic absorption. The film compositions of the present invention are particularly useful for whitening tooth surfaces. Generally, the delivery of the peroxide, with or without additional actives, involves topically applying the inventive film containing a safe and containing effective amount of such compounds/actives to a tooth or teeth and gums in a manner described in U.S. Pat. Nos. 5,894,017; 5,891,453; 6,045,811; and 6,419,906, each of which is herein incorporated by reference in its entirety. The frequency of application and the period of use will vary widely depending upon the level of treatment required or desired, e.g., the degree of teeth whitening and/or degree of topical wound healing/disinfection desired.

### EXAMPLES

The film compositions illustrated in following examples illustrate specific embodiments of the film compositions of the present invention, but are not intended to be limiting thereof. Other modifications can be undertaken by the skilled artisan without departing from the spirit and scope of this invention.

All exemplified film compositions can be prepared by conventional formulation and mixing techniques. Component amounts are listed as weight percents and exclude minor materials such as diluents, filler, and so forth. The listed formulations, therefore, comprise the listed components and any minor materials associated with such components.

### Example I

The following is an example of a bi-layer, teeth whitening film of the present invention.

| | AMOUNT | |
|---|---|---|
| INGREDIENT | (weight percent) | |
| Adhesive Layer | | |
| XANTHAN GUM¹ | 0.0174% | w/w |
| LOCUST BEAN GUM², CLARIFIED | 0.0348% | w/w |
| CARRAGEENAN³ | 0.1740% | w/w |
| PULLULAN⁴ | 4.1000% | w/w |
| POVIDONE, USP K-90⁵ | 12.4000% | w/w |
| SUCRALOSE⁶ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁷ | 5.7100% | w/w |
| FLAVOR | 2.5890% | w/w |
| POLYSORBATE 80 NF/EP⁸ | 0.3550% | w/w |
| EMULSIFIER⁹ | 0.3550% | w/w |
| GLYCERIN USP SPECIAL | 1.0000% | w/w |
| Backing Layer | | |
| PHARMACEUTICAL GLAZE, 4-LB CUT NF¹⁰ | 55.0000% | w/w |
| SILICA¹¹ (fumed untreated) | 4.0000% | w/w |
| ALCOHOL USP/EP | 40.0000% | w/w |
| GLYCERYL STEARATE SE¹² | 1.0000% | w/w |

¹Supplied under the name Keltrol T by CP Kelco, Chicago, IL
²Sold under the name Viscogum BCR 20/80 by Degussa Texturant Systems, Atlanta, GA
³Supplied under the name Viscarin SD339 by FMC Biopolymer, Philadelphia, PA.
⁴PI-20 grade supplied by Hayashibara.
⁵Polyvinylpyrrolidone, USP K-90, International Specialties Products(ISP), Wayne, NJ.
⁶ALB CG 35% hydrogen peroxide solution, Atofina, Philadelphia, Pa.
⁷Supplied under the trade name Splenda ®, by McNeil Pharmaceuticals, New Brunswick, NJ.
⁸Tween 80, supplied by Quest, Hoffmann Estates, Ill.
⁹mixture of mono- and di-oleates supplied under name Atmos 300 by American Ingredients, Kansas City, Mo.
¹⁰Shellac supplied by Mantrose Haeser Co., Attleboro, Ma.
¹¹Supplied under the trade name Cabosil ® by Cabot, Tuscola, Ill.
¹²Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, locust bean gum, carrageenan, pullulan and Povidone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example II

The following is an example of a bi-layer, teeth whitening film of the present invention.

| | AMOUNT | |
|---|---|---|
| INGREDIENT | (weight percent) | |
| Adhesive Layer | | |
| XANTHAN GUM¹ | 0.02308% | w/w |
| LOCUST BEAN GUM², CLARIFIED | 0.04616% | w/w |
| CARRAGEENAN³ | 0.2308% | w/w |
| POVIDONE, USP K-90⁴ | 16.426% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |
| Backing Layer | | |
| PHARMACEUTICAL GLAZE, 4-LB CUT NF⁹ | 55.0000% | w/w |
| SILICA¹⁰ (fumed untreated) | 4.0000% | w/w |
| ALCOHOL USP/EP | 40.0000% | w/w |
| GLYCERYL STEARATE SE¹¹ | 1.0000% | w/w |

¹Supplied under the name Keltrol T by CP Kelco, Chicago, IL
²Supplied under the name Viscogum BCR 20/80 by Degussa Texturant Systems, Atlanta, GA
³Supplied under the name Viscarin SD339 by FMC Biopolymer, Philadelphia, PA.
⁴Polyvinylpyrrolidone, USP K-90, International Specialties Products(ISP), Wayne, NJ.
⁵ALB CG 35% hydrogen peroxide solution, Atofina, Philadelphia, Pa.
⁶Supplied under the trade name Splenda ®, by McNeil Pharmaceuticals, New Brunswick, NJ.
⁷Tween 80, supplied by Quest, Hoffmann Estates, Ill.
⁸mixture of mono- and di-oleates supplied under name Atmos 300 by American Ingredients, Kansas City, Mo.
⁹Shellac supplied by Mantrose Haeser Co., Attleboro, Ma.
¹⁰Supplied under the trade name Cabosil ® by Cabot, Tuscola, Ill.
¹¹Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, locust bean gum, carrageenan and Povidone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example III

The following is an example of a bi-layer, teeth whitening film of the present invention.

| | AMOUNT | |
|---|---|---|
| INGREDIENT | (weight percent) | |
| Adhesive Layer | | |
| XANTHAN GUM¹ | 0.0674% | w/w |
| LOCUST BEAN GUM, CLARIFIED² | 0.0848% | w/w |
| PULLULAN³ | 4.1740% | w/w |
| POVIDONE, USP K-90⁴ | 12.4000% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |
| Backing Layer | | |
| PHARMACEUTICAL GLAZE, 4-LB CUT NF⁹ | 55.0000% | w/w |
| SILICA¹⁰(fumed untreated) | 4.0000% | w/w |
| ALCOHOL USP/EP | 40.0000% | w/w |
| GLYCERYL STEARATE SE¹¹ | 1.0000% | w/w |

¹Supplied under the name Keltrol T by CP Kelco, Chicago, IL
²Supplied under the name Viscogum BCR 20/80 by Degussa Texturant Systems, Atlanta, GA
³PI-20 grade supplied by Hayashibara.
⁴Polyvinylpyrrolidone, USP K-90, International Specialties Products(ISP), Wayne, NJ.
⁵ALB CG 35% hydrogen peroxide solution, Atofina, Philadelphia, Pa.
⁶Supplied under the trade name Splenda ®, by McNeil Pharmaceuticals, New Brunswick, NJ.
⁷Tween 80, supplied by Quest, Hoffmann Estates, Ill.
⁸mixture of mono- and di-oleates supplied under name Atmos 300 by American Ingredients, Kansas City, Mo.
⁹Shellac supplied by Mantrose Haeser Co., Attleboro, Ma.
¹⁰Supplied under the trade name Cabosil ® by Cabot, Tuscola, Ill.
¹¹Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, locust bean gum, pullulan and povidone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example IV

The following is an example of a bi-layer, teeth whitening film of the present invention.

| | AMOUNT | |
|---|---|---|
| INGREDIENT | (weight percent) | |
| Adhesive Layer | | |
| PECTIN¹ | 1.9674% | w/w |
| GUM ARABIC² | 0.1848% | w/w |
| PULLULAN³ | 2.1740% | w/w |
| POVIDONE, USP K-90⁴ | 12.4000% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |
| Backing Layer | | |
| PHARMACEUTICAL GLAZE, 4-LB CUT NF⁹ | 55.0000% | w/w |
| SILICA¹⁰(fumed untreated) | 4.0000% | w/w |
| ALCOHOL USP/EP | 40.0000% | w/w |
| GLYCERYL STEARATE SE¹¹ | 1.0000% | w/w |

¹Supplied under the name GenuPectin by CP Kelco, Chicago, IL.
²Supplied under the name Bright Gum Arabic Spray Dry FCC/NF Powder by TIC Gums, Belcamp, MD.
³PI-20 grade supplied by Hayashibara.
⁴Polyvinylpyrrolidone, USP K-90, International Specialties Products(ISP), Wayne, NJ.
⁵ALB CG 35% hydrogen peroxide solution, Atofina, Philadelphia, Pa.
⁶Supplied under the trade name Splenda ®, by McNeil Pharmaceuticals, New Brunswick, NJ.
⁷Tween 80, supplied by Quest, Hoffmann Estates, Ill.
⁸mixture of mono- and di-oleates supplied under name Atmos 300 by American Ingredients, Kansas City, Mo.
⁹Shellac supplied by Mantrose Haeser Co., Attleboro, Ma.
¹⁰Supplied under the trade name Cabosil ® by Cabot, Tuscola, Ill.
¹¹Supplied as Mono- and Diglycerides of fats and oils (disposablegrade) by Lonza Inc., Fair Lawn, NJ.

In a suitable beaker (beaker A), water, sucralose, potassium phosphate monobasic are added with mixing until the mixture is homogenous.

In a separate beaker (beaker B), starch gum, gum arabic, pullulan and povidone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example V

The following is an example of a bi-layer, teeth whitening film of the present invention.

| | AMOUNT | |
|---|---|---|
| INGREDIENT | (weight percent) | |
| Adhesive Layer | | |
| XANTHAN GUM¹ | 0.0174% | w/w |
| GUM ARABIC² | 0.1848% | w/w |
| PULLULAN³ | 16.344% | w/w |
| CARRAGEENAN⁴ | 0.1800% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |
| Backing Layer | | |
| PHARMACEUTICAL GLAZE, 4-LB CUT NF⁹ | 55.0000% | w/w |
| SILICA¹⁰ (fumed untreated) | 4.0000% | w/w |
| ALCOHOL USP/EP | 40.0000% | w/w |
| GLYCERYL STEARATE SE¹¹ | 1.0000% | w/w |

¹Supplied under the name Keltrol T by CP Kelco, Chicago, IL
²Supplied under the name Bright Gum Arabic Spray Dry FCC/NF Powder by TIC Gums, Belcamp, MD
³PI-20 grade supplied by Hayashibara.
⁴Supplied under the name Viscarin SD339 by FMC Biopolymer, Philadelphia, PA.
⁵Supplied under the trade name Splenda ®, by McNeil Pharmaceuticals, New Brunswick, NJ.
⁶ALB CG 35% hydrogen peroxide solution, Atofina, Philadelphia, Pa.
⁷Tween 80, supplied by Quest, Hoffmann Estates, Ill.
⁸mixture of mono- and di-oleates supplied under name Atmos 300 by American Ingredients, Kansas City, Mo.
⁹Shellac supplied by Mantrose Haeser Co., Attleboro, Ma.
¹⁰Supplied under the trade name Cabosil ® by Cabot, Tuscola, Ill.
¹¹Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, gum arabic, pullulan, and carrageenan are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example V

The following is an example of a bi-layer, teeth whitening film of the present invention.

| | AMOUNT | |
|---|---|---|
| INGREDIENT | (weight percent) | |
| Adhesive Layer | | |
| XANTHAN GUM¹ | 0.0174% | w/w |
| GUM ARABIC² | 0.1848% | w/w |
| STARCH³ | 16.344% | w/w |
| CARRAGEENAN⁴ | 0.1800% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |
| Backing Layer | | |
| PHARMACEUTICAL GLAZE, 4-LB CUT NF⁹ | 55.0000% | w/w |
| SILICA¹⁰ (fumed untreated) | 4.0000% | w/w |
| ALCOHOL USP/EP | 40.0000% | w/w |
| GLYCERYL STEARATE SE¹¹ | 1.0000% | w/w |

¹Supplied under the name Keltrol T by CP Kelco, Chicago, IL
²Supplied under the name Bright Gum Arabic Spray Dry FCC/NF Powder by TIC Gums, Belcamp, MD
³Supplied under the trade name of Pure-Cote B760, supplied by Grain processing Corporation, Muscatine, IA.
⁴Supplied under the name Viscarin SD339 by FMC Biopolymer, Philadelphia, PA.
⁵Supplied under the trade name Splenda ®, by McNeil Pharmaceuticals, New Brunswick, NJ.
⁶ALB CG 35% hydrogen peroxide solution, Atofina, Philadelphia, Pa.
⁷Tween 80, supplied by Quest, Hoffmann Estates, Ill.
⁸mixture of mono- and di-oleates supplied under name Atmos 300 by American Ingredients, Kansas City, Mo.
⁹Shellac supplied by Mantrose Haeser Co., Attleboro, Ma.
¹⁰Supplied under the trade name Cabosil ® by Cabot, Tuscola, Ill.
¹¹Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, gum arabic, starch, and carrageenan are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

1. An oral composition comprising:
a.) at least two water soluble gums; and
b.) at least one peroxide compound
wherein the composition, when dried, contains less than about 10% of an hydroalcoholic component.

2. An oral composition according to claim 1, wherein the concentration of the at least two water soluble polymers is from about 0.01% to about 10%.

3. An oral composition according to claim 1, wherein the concentration of the at least one peroxide compound is from about 0.1% to about 20%.

4. An oral composition according to claim 1 wherein the water soluble gum is selected from the group consisting of algin, alginic acid, alginate salts, camitine, carrageenan, dextrin, karaya gum , guar gum, gellan gum, irish moss, veegum (regular), tara gum, okra gum, gum arabic, acacia gum, amylopectin, pectina or pecitin, ghatti gum, natto gum, tragacanth gum, xanthan gum, sclerotium gum, kelp, locust bean gum, psyllium seed, tamarind gum, destria gum, chitosan, esters thereof, salts thereof and mixtures thereof.

5. An oral composition according to claim 4 wherein the water soluble gum is xanthan gum, locust bean gum, carrageenan, dextrin, gum arabic and mixtures thereof.

6. An oral composition according to claim 1 wherein the peroxide compound is selected from the group consisting of hydrogen peroxide, calcium peroxide, urea peroxide, carbamide peroxide, and mixtures thereof.

7. An oral composition according to claim 6 wherein the peroxide is hydrogen peroxide.

8. An oral composition according to claim 1 further comprising an active agent wherein the topical or systemic active is selected from the group consisting of additional whitening agents, antitartar agents, fluoride ion sources, antimicrobial agents, anesthetic agents and mixtures thereof.

9. A mono- or multi-layer film composition comprising at least one layer comprising:
a.) at least two water soluble gums and
b.) at least one peroxide compound
wherein the composition, when dried, contains less than about 10% hydroalcoholic components.

10. A film composition according to claim 9 wherein the water soluble gum is selected from the group consisting of algin, alginic acid, alginate salts, camitine, carrageenan, dextrin, karaya gum, guar gum, gellan gum, irish moss, veegum (regular), tara gum, okra gum, gum arabic, acacia gum, amylopectin, pectina or pecitin, ghatti gum, natto gum, tragacanth gum, xanthan gum, sclerotium gum, kelp, locust bean gum, psyllium seed, tamarind gum, destria gum, chitosan, esters thereof, salts thereof and mixtures thereof.

11. A film composition according to claim 10 wherein the peroxide is xanthan gum, locust bean gum, carrageenan, dextrin, gum arabic and mixtures thereof.

12. A film composition according to claim 9 wherein the peroxide compound is selected from the group consisting of hydrogen peroxide, calcium peroxide, urea peroxide, carbamide peroxide, and mixtures thereof.

13. A film composition according to claim 12 wherein the peroxide is hydrogen peroxide.

14. An oral composition comprising:
a.) at least two water soluble gums; and
b.) at least one peroxide compound
wherein the composition, when dried, contains less than about 10% of an hydroalcoholic component and wherein the ratio of the water soluble gum to peroxide is at least about 1:25.

15. An oral composition according to claim 14, wherein the ratio of water soluble gum to peroxide is from about 1:25 to about 1:5.

16. A method of whitening teeth comprising the step of applying to the teeth the oral composition of claim 1.
